(19) United States
(12) Patent Application Publication
(10) Pub. No.: US 2024/0130897 A1
(43) Pub. Date: Apr. 25, 2024

(54) WOUND DRESSING SYSTEMS AND METHODS OF USE

(71) Applicant: 145 LTD., Spruce Grove (CA)

(72) Inventor: Stephen FERRY, Spruce Grove (CA)

(73) Assignee: 145 LTD., Spruce Grove, AB (CA)

(21) Appl. No.: 18/546,819

(22) PCT Filed: Mar. 4, 2021
(86) PCT No.: PCT/CA2021/050285

§ 371 Date: Aug. 16, 2023

(30) Foreign Application Priority Data
Feb. 17, 2021 (CA) 3109057

(51) Int. Cl. A61F 13/0246; A61F 13/00; A61F 13/0203; A61F 13/14

(52) U.S. Cl. CPC A61F 13/0253; A61F 13/00076; A61F 13/0213; A61F 13/14

(57) ABSTRACT

A wound dressing system has a dressing. The dressing may have a bandage; and the dressing may form a patient-contacting face that defines an interior wound-contacting portion and a peripheral adhesive portion, the peripheral adhesive portion at least partially surrounding the interior wound-contacting portion, with a removable outer cover overlying the peripheral adhesive portion. A method involves securing the wound dressing system over a wound on a patient.

### TECHNICAL FIELD

This document relates to wound dressing systems and methods of use.

### BACKGROUND

The following paragraphs are not an admission that anything discussed in them is prior art or part of the knowledge of persons skilled in the art.

U.S. Pat. No. 5,628,723 discloses a bandage denoted an Emergency Bandage, also known as an Israeli Bandage, that comprises a sterile dressing held in place over a wound by a web portion, and a pressure enhancement member that transfers a force from the web portion to the wound via the dressing.

U.S. Pat. No. 7,652,190 discloses a wound dressing system comprising an elongated elastic wrap in which is a provided a receptacle for storing treatment material, for example sterile fabric or gauze. The system also comprises a plastic occlusion layer. This wound dressing system comprises a substantially rigid pressure member with the approximate shape of a dome, which can optionally be used as an eye cover.

One commercial wound dressing system, the SAM Chest Seal™, comprises a patch of impermeable material that adheres to the skin surrounding a wound using a hydrogel adhesive. The Chest Seal may further comprise a plastic one-way valve that allows air egress from the wound but prevents ingress. Asherman™ also sells a chest seal that is similar to the SAM Chest Seal.

### SUMMARY

A wound dressing system is disclosed comprising a dressing. The dressing may have a bandage; and the dressing may form a patient-contacting face that defines an interior wound-contacting portion and a peripheral adhesive portion, the peripheral adhesive portion at least partially surrounding the interior wound-contacting portion, with a removable outer cover overlying the peripheral adhesive portion.

A method is disclosed comprising securing the wound dressing system over a wound on a patient.

A wound dressing system is disclosed comprising: a dressing; and a bandage secured to the dressing, the bandage having an adhesive tail portion comprising hydrogel.

A wound dressing system is disclosed, comprising: a dressing forming a patient-contacting face that defines an interior wound-contacting portion and a peripheral adhesive portion, the peripheral adhesive portion at least partially surrounding the interior wound-contacting portion, with a removable outer cover overlying the peripheral adhesive portion, the removable outer cover having a dressing pad.

The interior wound-contacting portion comprises a dressing pad.

A wound dressing system is disclosed, comprising: a bandage, having a length of at least forty eight inches; and a dressing.

A wound dressing system is disclosed, comprising: a dressing forming a patient-contacting face that defines an interior wound-contacting portion and a peripheral adhesive portion, the peripheral adhesive portion comprising hydrogel and at least partially surrounding the interior wound-contacting portion, with a removable outer cover overlying the peripheral adhesive portion, and the interior wound-contacting portion having a non-adherent gauze dressing pad.

A wound dressing system is disclosed comprising: a bandage; and a dressing having: an adhesive layer; a removable outer cover layer overlying the adhesive layer; and a dressing pad layer overlying the removable outer cover layer; in which the removable outer cover layer and the dressing pad layer are cut along aligned interior paths to separate the removable outer cover layer and the dressing pad layer into interior and peripheral portions.

In various embodiments, there may be included any one or more of the following features: The removable outer cover comprises a dressing pad. The interior wound-contacting portion comprises a dressing pad. The dressing pad comprises a non-adherent sterile gauze pad. The dressing is formed by: an adhesive layer defining the peripheral adhesive portion; a removable outer cover layer defining the removable outer cover; and a dressing pad layer overlying the removable outer cover layer; in which the removable outer cover layer and the dressing pad layer are cut along aligned interior paths to separate the removable outer cover layer and the dressing pad layer into interior and peripheral portions, with the interior portion of the dressing pad layer defining the interior wound-contacting portion, and the peripheral portion defining a peripheral wound-contacting portion. The peripheral adhesive portion comprises hydrogel. The peripheral adhesive portion fully surrounds the interior wound-contacting portion to form an occlusive dressing. A vent is defined in the dressing. The removable outer cover comprises a tab. The interior wound-contacting portion has a stadium shape. The bandage comprises elastomeric material. The bandage is sized to extend at least one revolution around a patient's torso. The bandage has a length of at least forty eight inches. The bandage has a length of at least sixty inches. The bandage has a detachable connector that secures the bandage to the dressing. The detachable connector comprises a frangible portion of the bandage. The detachable connector forms a hook and loop connector. The bandage comprises a dressing pad. The bandage is secured in an at least partially rolled configuration via a roll retention mechanism. The roll retention mechanism comprises a plurality of stays spaced longitudinally along the bandage. The plurality of stays comprise hook and loop connectors. The roll retention mechanism comprises a thread. The thread extends perpendicular relative to a roll axis of the bandage in the at least partially rolled configuration. The thread comprises adhesive. The bandage has an adhesive tail portion. The adhesive tail portion comprises hydrogel. The adhesive tail portion has a removable outer tail portion cover. The removable outer tail portion cover comprises a tab. An outer packaging enclosing the bandage and dressing portion. The outer packaging comprises vacuum packaging. The interior wound-contacting portion is sized to cover a bullet wound. The interior wound-contacting portion has a minimum lateral dimension of two inches or more. The system is secured to a patient, in which the interior wound-contacting portion overlies a bullet or shrapnel wound on the patient.

The foregoing summary is not intended to summarize each potential embodiment or every aspect of the subject matter of the present disclosure. These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1 is an isometric view of a wound dressing system in a vacuum-sealed package.

FIG. 2 is a side elevation view of the wound dressing system of FIG. 1 removed from the packaging.

FIG. 3 is an exploded isometric view of a multi-layer dressing.

FIG. 4 is an exploded isometric view of a multi-layer adhesive tail portion for a bandage.

FIG. 5 is a top plan view of a wound dressing system incorporating a bandage, as well as the multi-layer dressing and adhesive tail portion from FIGS. 3 and 4, respectively.

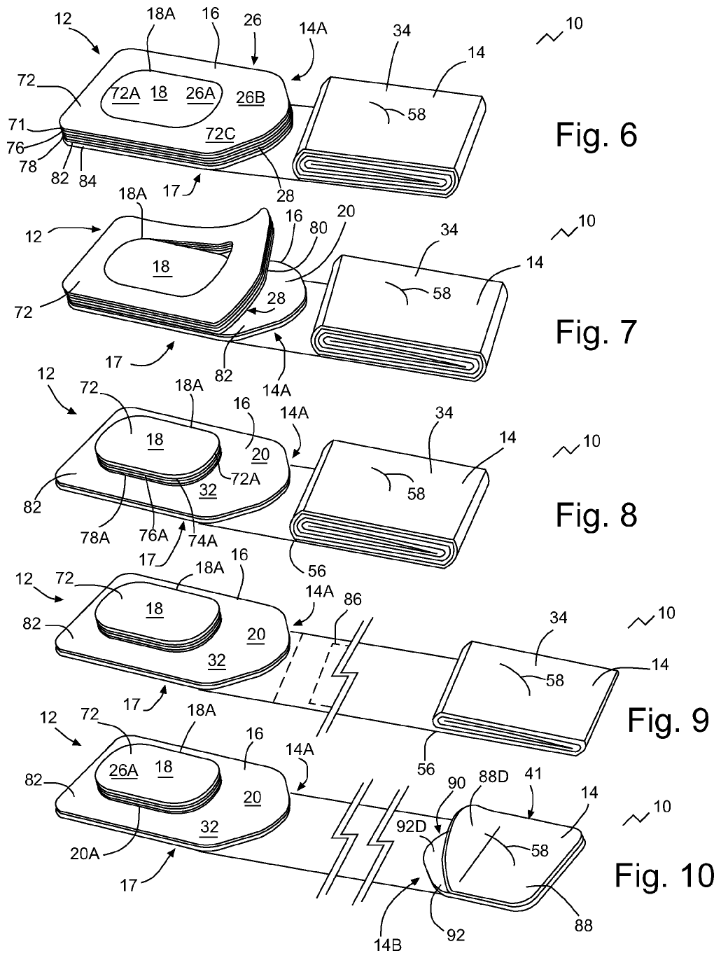

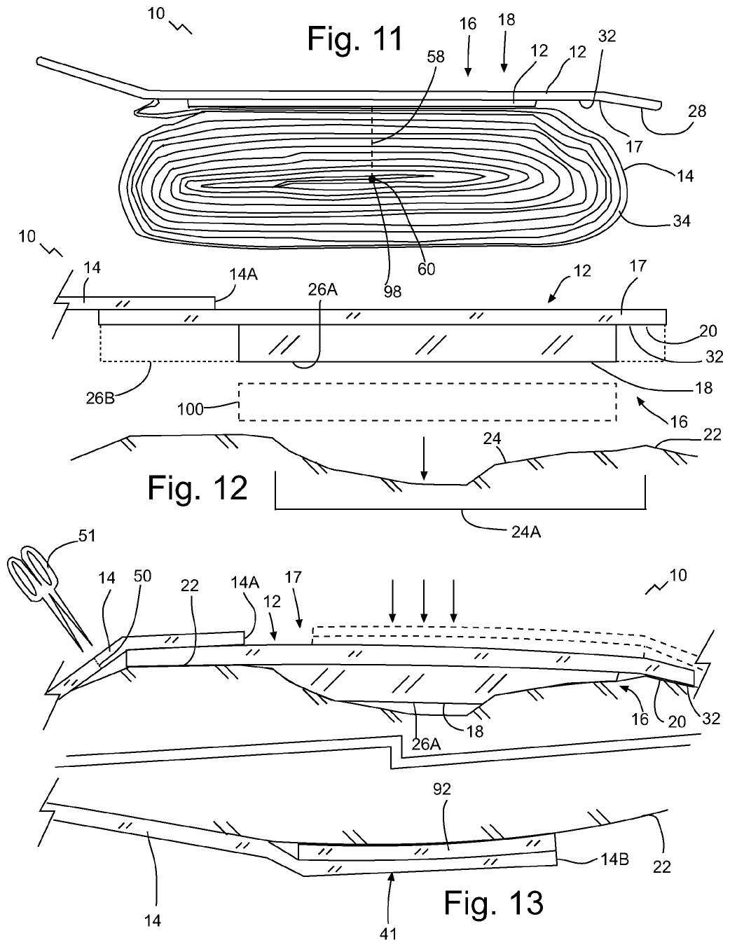

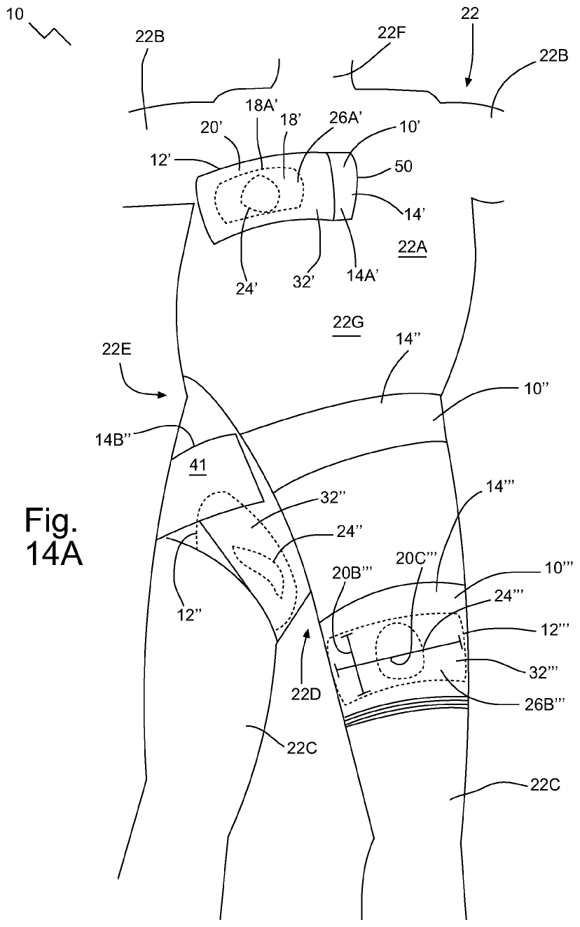

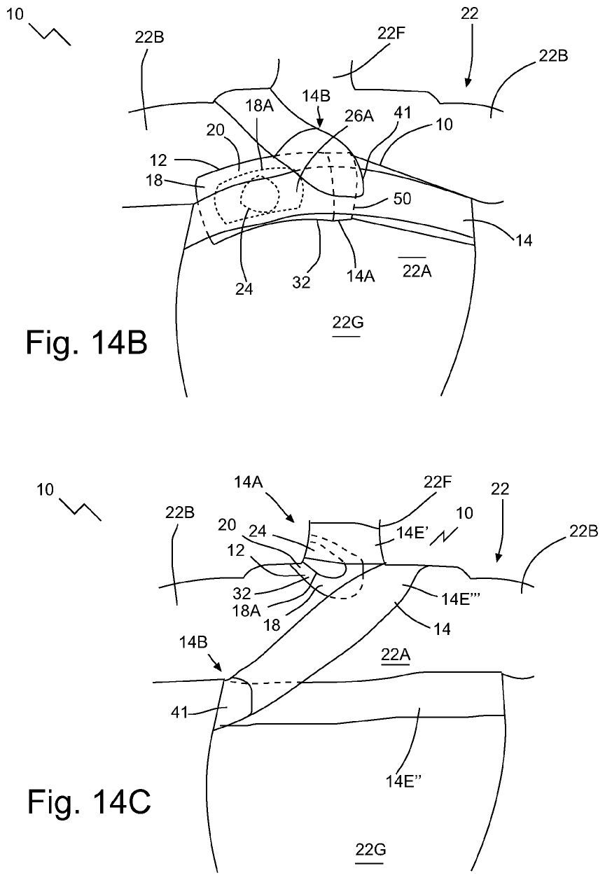

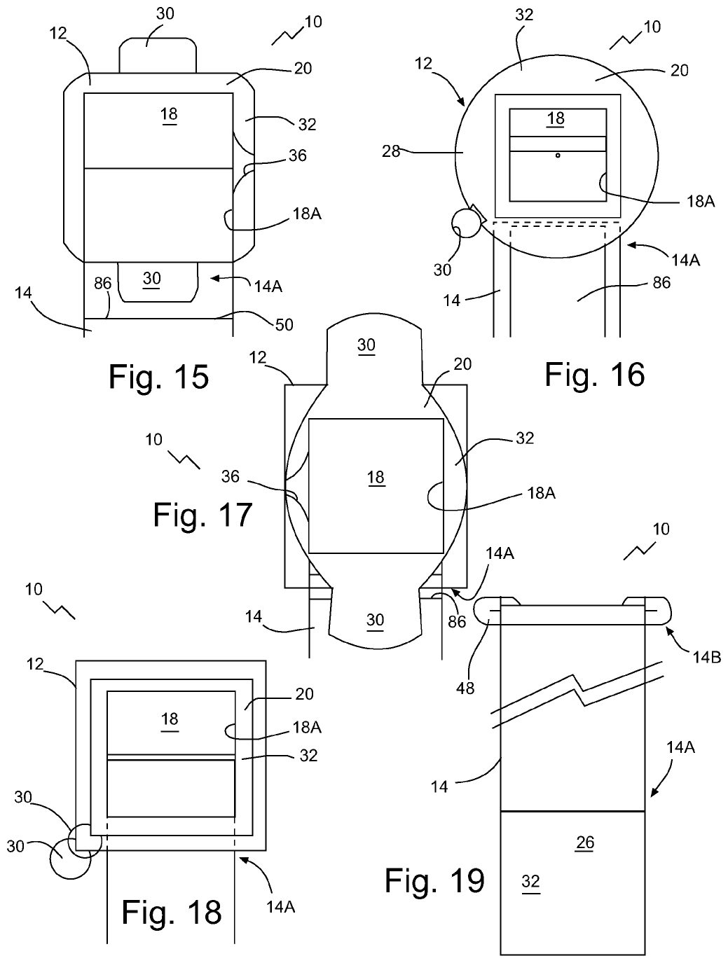

FIGS. 6-10 are isometric views of the wound dressing system of FIG. 5 shown in various states in a progressive sequence, for example:
i. FIG. 6—the system is out of the package with the dressing unrolled from the bandage;
ii. FIG. 7—the tab of a removable outer cover is lifted to begin to peel away the removable outer cover from a peripheral adhesive portion of the dressing;
iii. FIG. 8—the removable outer cover is removed, leaving an interior wound-contacting portion of the dressing, and the peripheral adhesive portion exposed;
iv. FIG. 9—the bandage is partially unrolled, with a roll retention mechanism restraining the rolled portion of the bandage; and
v. FIG. 10—the bandage is fully unrolled to reveal the adhesive tail portion, and a tab of a removable outer tail portion cover of the adhesive tail portion is lifted to begin to peel away the removable outer tail portion cover.

FIG. 11 is a side elevation view of the wound dressing system of FIG. 5 in a rolled state after removal from packaging.

FIGS. 12 and 13 are side elevation cross-section views of a wound dressing system being applied to a wound on a patient.

FIG. 14A is a front elevation view of a patient, with the wound dressing system applied in different ways to a chest, groin, and extremity (leg) of the patient.

FIG. 14B is a front elevation view of an upper torso of a patient, with the wound dressing system applied to a chest injury, with the bandage wrapped around the upper chest and shoulders.

FIG. 14C is a front elevation view of an upper torso of a patient, with the wound dressing system applied to a neck injury, with the bandage wrapped around the neck, upper chest, shoulders.

FIGS. 15-19 are top plan views of a variety of embodiments of wound dressing systems.

### DETAILED DESCRIPTION

In a combat or emergency situation, a victim may be inflicted with one or more severe open wounds that put the patient at risk of severe bleeding, hemorrhage and shock. Acute bleeding is a frequent yet entirely preventable cause of death in a combat or emergency/trauma situation. In such an event, the primary objective is to stem the flow of blood as soon as possible. Depending on the severity and nature of the wound, a patient may have a window of seconds to minutes during which an effective dressing must be applied over the wound in a suitable manner in order to save the patient's life from excessive blood loss. A patient is often unable to treat themselves, either due to wound access issues, shock, lack of consciousness or lucidity, or other issues. Thus, a wound will often need to treated by a medic or emergency personnel by the temporary application of a wound dressing. A wound dressing is placed in direct contact with the wound and may serve to maintain pressure on blood vessels to limit bleeding, prevent contamination of the wound by bacteria and debris, or prevent further injury, swelling, or painful stimuli.

The proper application of an effective wound dressing is the first stage in the healing process of a severe wound. In addition to the dangers of excessive blood loss, an open wound is at risk of being contaminated with foreign matter and bacteria, and is thus prone to subsequent infection. The treatment of a wound in a hospital setting generally involves cleaning and/or removal of foreign matter and closure, often with stitches or other skin binding techniques. The first stage of wound care intends to achieve hemostasis or clotting of the blood, which occurs a few minutes after injury as platelets in the blood begin to stick to the injured site, triggering the polymerization of fibrin, a protein, which forms a barrier to prevent further bleeding from the damaged blood vessel. In the process of wound healing, a complex sequence of cell proliferation and specialization then proceeds to repair and replace the damaged tissue. A suitable wound dressing creates a barrier that prevents blood loss and facilitates the natural clotting process.

Combat wounds are a particular type of wound that occur on the battlefield. Combat wound care has evolved immensely throughout the years in the military arena. Combat wounds may present complex challenges in the field of wound care, due to depth and extent of laceration and penetrating wounds, and due to a risk of relatively higher contamination as a result of being in the environment where the wound occurred, i.e. the battlefield. Common combat wounds experienced on the modern battlefield include: a) blast wounds—for example from landmines, grenades, IEDs (improvised explosive devices), and suicide bombings, with amputation rates being high for such wounds, b) gunshot and shrapnel wounds, causing deep and bloody lacerations; and c) head injuries and fractured bones.

There are numerous challenges in managing combat wounds. The challenge and complexity of combat wounds often include relatively large wound size and heavy amount of drainage. The current standard of care when working with a combat wound may involve using impregnated polyhexamethylene biguanide gauze dressings, silver dressings—such as containing antimicrobial properties, negative pressure wound therapy—which may require less frequent dressing changes and controls high amounts of exudate, and moisture sensors—which allow dressing decisions without disturbing the dressing. Debridement in combat wounds also presents a challenge to combat wound care. Combat wounds must be debrided aggressively, whether dealing with bony or soft tissue. Debridement must be ideally performed as soon as possible and under magnification using surgical operating loupes. High-pressure water jets and surgical instruments may be used in combination to clean bone and soft tissues. Nutritional challenges also often arise in combat wound situations. Soldiers who have been deployed for a longer duration are more likely to be malnourished at the time of traumatic injury. Enteral feeding is implemented for soldiers with complex injuries.

Combat wounds are more likely to be severe and to target the chest and junction areas. Adequate pre-hospital care may be more critical to the survival of the victim in such situations, since access to hospitals may be limited and supplies may be limited. Quickly achieving hemostasis in a wounded combat victim may be crucial to the patients survival. Combat wound dressing systems may comprise bandages, dressing pads, and other accessories for treating wound victims in the pre-hospital setting, for example combat situations or civilian emergencies. Combat wound dressing systems may be constructed to minimize human error when they are used in emergency situations when both victim and medic are in a state of stress or shock and time is of the essence. Traditional dressings are unable to secure packing material while maintaining pressure and multiple adjuncts are often required to treat a single wound. Common combat junctional wounds occur at the scalp, neck, axilla, inguinal and perineum. The three primary preventable causes of death on the battlefield include 1) bleeding from an extremity wound, 2) tension pneumothorax, and 3) airway obstructions.

A dressing may include a sterile pad or compress applied to a wound to promote healing and protect the wound from further harm. A dressing may be designed to be in direct contact with the wound, as distinguished from a bandage, which is most often used to hold a dressing in place. Many modern dressings are self-adhesive.

A dressing may have a number of purposes, depending on the type, severity and position of the wound, although all purposes are focused on promoting recovery and protecting from further harm. Various purposes of a dressing may include:
i. Stem bleeding—to help to seal the wound to expedite the clotting process;
ii. Protection from infection—to defend the wound against germs and mechanical damage;
iii. Absorb exudate—to soak up blood, plasma, and other fluids exuded from the wound, containing it/them in one place and preventing maceration;
iv. Ease pain—either by a medicated analgesic effect, compression or simply preventing pain from further trauma;
v. Debride the wound—to remove slough and foreign objects from the wound to expedite healing;
vi. Restore chest wall integrity—seals off chest wounds to permit proper or functional breathing capacity; and
vii. Reduce psychological stress—to obscure a healing wound from the view of the patient and others.

Ultimately, the aim of a dressing is to promote healing of the wound by providing a sterile, breathable and moist environment that facilitates granulation and epithelialization. This will then reduce the risk of infection, help the wound heal more quickly, and reduce scarring.

Historically, a dressing was made of a piece of material, usually a cloth, but the use of cobwebs, dung, leaves and honey have also been described. However, a modern dressing may include dry or impregnated gauze, plastic films, gels, foams, hydrocolloids, alginates, hydrogels, and polysaccharide pastes, granules and beads. Such examples may provide different physical environments suited to different wounds such as:
i. Absorption of exudate, to regulate the moisture level surrounding the wound—for example, dry gauzes absorb exudate strongly, drying the wound, while hydrocolloids maintain a moist environment, and film dressings do not absorb exudate;
ii. Gas permeability and exchange, especially with regard to oxygen and water vapour;
iii. Maintaining optimum temperature to encourage healing;
iv. Mechanically debriding a wound to remove slough; and
v. Pressure dressings are commonly used to treat burns and after skin grafts. They apply pressure and prevent fluids from collecting in the tissue.

A dressing may be held in place by a bandage. A bandage may comprise a piece of material used either to support a medical device such as a dressing or splint, or used on its own to provide support to or to restrict the movement of a part of the body. When used with a dressing, the dressing may be applied directly on a wound, and a bandage used to hold the dressing in place. Other bandages may be used without dressings, such as elastic bandages that are used to reduce swelling or provide support to a sprained ankle. Tight bandages can be used to slow blood flow to an extremity, such as when a leg or arm is bleeding heavily. Bandages are available in a wide range of types, from generic cloth strips to specialized shaped bandages designed for a specific limb or part of the body. Bandages can often be improvised as the situation demands, using clothing, blankets or other material. A bandage may include a relatively long strip of woven and/or elastic material, for example cotton or polyester, that can be wrapped around a victim's limb, torso, neck or otherwise used to hold a dressing in place. A bandage may be wrapped tightly to apply compression to a wound. Some dressings are self-adhesive and do not require bandages, which may be advantageous. It may, for example, be difficult to use a bandage to secure a dressing to a junction wound, which is a wound located at the armpit (axillar wounds), groin, neck, scalp, or perineum.

An elastic bandage may be used. An elastic bandage may comprise a stretchable bandage used to create localized pressure. Elastic bandages are commonly used to treat muscle sprains and strains by reducing the flow of blood to a particular area by the application of even stable pressure which can restrict swelling at the place of injury. Elastic bandages are also used to treat bone fractures. Padding is applied to the fractured limb, after which a splint (usually plaster) is applied. The elastic bandage may then be applied to hold the splint in place and to protect it. This is a common technique for fractures which may swell, which would cause a cast to function improperly. These types of splints are usually removed after swelling has decreased and then a fiberglass or plaster cast can be applied.

Referring to FIG. 5, a wound dressing system 10 comprises a dressing 12. In some cases the dressing 12 includes a bandage 14. The dressing 12 and bandage 14 may be secured together by a suitable method, such as using one or more of adhesive, stitches, rivets, clasps, or integral formation. Referring to FIG. 14, in use, the patient 22 or a third party (such as a medic) secures the wound dressing system 10 over a wound 24 on a patient 22. Referring to FIG. 15, the dressing 12 may have a removable outer cover 28 overlying a peripheral adhesive portion 20. the removable outer cover 28 may have a dressing pad, such as a non-adherent layer 72. Referring to FIG. 10, the bandage 14 may have an adhesive tail portion 41. Referring to FIG. 14, the bandage 14 may have a length sufficient to wrap at least once around a torso of a patient 22, for example if the bandage 14 has a length of at least forty eight inches, for example at least seventy two inches. The dressing 12 may comprise hydrogel adhesive. Referring to FIG. 3, the dressing 12 may have a multi-layer configuration adapted for a plurality of single, dual, triple, or more uses, thus forming a compact, flexible, and effective tool on the battlefield and the emergency context.

Referring to FIGS. 6-10, the wound dressing system 10 may comprise a bandage 14, for example secured to the dressing 12. A bandage 14 may have a plurality of uses in the field. A bandage 14 may be used to apply pressure to a wound to help stem bleeding. For extremity wounds in particular, hemostasis may be achieved by wrapping a bandage tightly around the wound to apply pressure to the wound. A bandage 14 used to apply compression may also minimize inflammation and further pain, or prevent accumulation of fluid at the wound site, for example in the case of a burn wound. A bandage 14 may also be used to create a tourniquet by tightly winding the bandage 14 near a wound to constrict the flow of blood to the wound. A bandage 14 may be constructed from a suitable material, such as an elastic, flexible, elastomeric material, so that the bandage 14 may conform to the shape of the patient, permit limited movement, and maintain tension and friction against the body, preventing the bandage and dressing from being dislodged. Referring to FIG. 3, the bandage 14 may be an elongate strap, such as an elastic strap as shown. The bandage 14 may have a first or dressing end 14A, a second or tail end 14B, and may define a length 14C and width 14D of suitable dimensions.

Referring to FIGS. 3, 6-8 and 12-13, a wound dressing system 10 may comprise a dressing 12 with a removable outer cover 28. The dressing 12 may form a patient-contacting face 16 that defines an interior wound-contacting portion 18 and a peripheral adhesive portion 20. The peripheral adhesive portion 20 may at least partially surround the interior wound-contacting portion 18, with a removable outer cover overlying the peripheral adhesive portion. In some cases such as shown, the peripheral adhesive portion 20 fully surrounds the interior wound-contacting portion 18 to form an occlusive dressing. The interior wound-contacting portion 18 may have a suitable shape, such as a stadium shape as shown, although other shapes may be used, including a rectangle (with or without rounded corners), a circle, an oval, a trapezoid, a polygon, and more complex shapes, including compound shapes having parts of one or more of the aforementioned, for example whose peripheral profile includes straight and/or curved lines.

Referring to FIGS. 3, 6-8 and 12-13, the removable outer cover 28 may form part of a peel and stick system. A peel-and-stick system may include an adhesive, such as portion 20, that can be protected before use by a removable backing that can easily be removed from said backing, but that provides a useful degree of adhesion to a target surface such as wound 24. In the example shown the adhesives comprises hydrogel.

Referring to FIGS. 3, 6-8 and 12-13, the interior wound-contacting portion 18 may comprise a dressing pad, such as a non-adherent sterile gauze pad. Such a pad may be provided as a non adherent layer 72. A dressing 12 may comprise one or more dressing pads, which may be sterile patches of material used to maintain the desired conditions at the wound Minimizing adherence of the dressing pad to the wound reduces pain when the dressing is removed, and also improves healing by preventing re-opening of the wound on removal. A dressing pad may also help debride the wound. Often, non-adherent dressings pads are made from loosely woven textiles such as cotton or synthetic gauze, which can absorb blood and wound exudate without sticking to the wound. This can help achieve hemostasis and maintain a stable blood clot at the wound. Gauze may be coated with a polymeric film to minimize sticking, and gauze dressings may also be impregnated with chemical agents that speed the natural clotting process.

In some cases wound packing may be used. For severe or deep wounds, wound packing may be used to absorb blood and exudate from within the wound, and may swell as it absorbs fluid to apply pressure from within the wound. Packing may be made from cotton or synthetic gauze. It may also be retained with a dressing and/or with a bandage. Like a dressing pad, packing may be impregnated with chemical agents that speed the natural clotting process.

Referring to FIGS. 3, 5, and 12-13, the interior wound-contacting portion 18 may be sized appropriately. In some cases, portion 18 may be sized to cover a bullet wound. The interior wound-contacting portion 18 may have a minimum lateral dimension, such as that of width 18B or length 18C of two inches or more, for example five inches or more. The lateral dimensions of portion 18 may be defined along the wound contacting face 16, and may extend between opposed edges of a window outline path 18A defining the periphery of the portion 18. The adhesive portion 20 may have a minimum lateral dimension, such as that of width 20B or length 20C defined along the wound contacting face 16, and may extend between opposed edges of a window outline path 20A defining the periphery of the portion 20.

Referring to FIGS. 3, 6-8 and 12-13, the dressing 12 may be structured to have the removal of the outer cover 28 to be optional. The outer cover 28 may be structured to contact a wound 24, for example if the cover 28 comprise a dressing pad, such as non-adherent sterile gauze pad. A medic, when using the system 10, may choose to remove or retain the removable outer cover 28 depending on the nature of the wound 24 to be treated. If the removable outer cover 28 comprises a dressing pad 26 and is left in place during treatment, the pad 26 may be in direct contact with a wound 24, for example a wound 24 that is almost as large as the wound-contacting face 16 of the dressing 12. Referring to FIG. 14A, thus, for a relatively small wound 24′, whose maximum lateral dimension is less than or equal to the minimum lateral dimensions of the portion 18, the cover 28 may be removed, the interior portion 18 aligned with the wound 24′, and the adhesive portion 20 applied around the periphery of the wound 24 to secure the dressing 12 to the wound 24. For a relatively large wound 24′″, whose maximum lateral dimension is greater than the minimum lateral dimensions of the portion 18, the cover 28 may be retainer, the interior portion 18 and dressing pad 26 aligned with the wound 24′″, and the dressing 12 secured to the wound 24 by wrapping the bandage 14 around the dressing 12 and patient 22.

Referring to FIGS. 3, 5-8, and 11, removable outer cover 28 may comprise a tab 30. The removable outer cover tab 30 may allow the removable outer cover 28 to be conveniently gripped and easily removed from the peripheral adhesive portion 20. The tab 30 may be formed by a sacrificial liner tab cover 80, which may be a plasticized or wax-coated sheet that provides a break between the adhesive portion 20 and a release line layer 78. The tab cover 80 may be sized and structured to permit a user to insert his or her fingers between the layer 78 and tab cover 80, permitting the user sufficient contact area to grip the layer 78 and tear the layer 78 off of the adhesive portion 20, exposing the portion 20. The tab cover 80 may oriented release layer down in the Figures.

Referring to FIGS. 3, 5-8, and 11, the dressing 12 may have multilayer form to provide the stated multifunctionality. The dressing 12 may be formed by an adhesive layer 82, a removable outer cover layer (paper release liner layer 78), and a dressing pad layer 72. The paper release line layer 82 may be positioned release side down in the figures. The adhesive layer 82 may define the peripheral adhesive portion 20. The removable outer cover layer 82 may overlie the removable outer cover or liner layer 78. The dressing pad layer 72 may overlie the removable outer cover layer 78. The removable outer cover layer 78 and the dressing pad 26/layer 72 may be cut along aligned interior paths 78A and 26A/72A, respectively, to separate the removable outer cover layer 78 and the dressing pad 26/layer 72 into the interior and peripheral portions 18 and 20, respectively.

Referring to FIGS. 3, 5-8, and 11, one or more other layers may be present in dressing 12. A pressure sensitive adhesive layer 76 may be present, for example seated with a base face 76G of layer 76 upon a top face 78F of layer 78. The layer 76 may be cut along aligned an interior path 76A that aligns with paths 78A and 26A, separating the layer 76 into interior and peripheral portions 76B and 76C, respectively. Pressure-sensitive adhesive (PSA, self-adhesive, self-stick adhesive) is a type of non-reactive adhesive which forms a bond when pressure is applied to bond the adhesive with a surface. No solvent, water, or heat is needed to activate the adhesive. It is used in pressure-sensitive tapes, labels, glue dots, note pads, automobile trim, and a wide variety of other products. A PSA may be designed to form a bond and hold properly at room temperatures. PSAs typically reduce or lose their tack at low temperatures and reduce their shear holding ability at high temperatures; special adhesives are made to function at high or low temperatures. A non-woven fabric layer 74 may be present, for example seated with a base face 74G of layer 74 upon a top face 76F of layer 76. The layer 75 may be cut along aligned an interior path 74A that aligns with paths 76A, 78A and 26A, separating the layer 74 into interior and peripheral portions 74B and 74C, respectively. Finally, layer 72 may be seated with a base face 72G of layer 72 upon a top face 74F of layer 74. The layer 72 may be cut along aligned an interior path 72A that aligns with paths 76A, 74A, 78A and 26A, separating the layer 72 into interior and peripheral portions 72B and 72C, respectively. A backing layer 84 may be provided, with the adhesive layer 82 seated with a base face 82G of layer 82 upon a top face 84F of layer 84. The layer 84 may be a hydrogel backing layer if hydrogel is the adhesive. A base face 84G of the layer 84 may be structured to grip the bandage 14, for example base face 84G may have a surface that grips or has relatively high friction with the bandage 14, forming a non-slip interface with the bandage 14 when the bandage 14 is pressed against the layer 84. In some cases the face 84G may comprise an adhesive, such as a peel-and-stick form to permit a release layer (not shown) to be removed to adhere the face 84G with the bandage 14. The layers may all have the same shape, such as if each layer has a corresponding nose (noses 72D, 74D, 76D, 78D, 80D, 82D, and 84G). The shape shown is that of a rounded edge rectangle with a rounded triangular nose, although other shapes for each layer may vary. Referring to FIG. 5, rounded triangular noses 80D and 90D are shown in solid lines, with alternative rectangular noses 80D′ and 90D′ shown in dashed lines as well. Rectangular dressings 12 and/or tail portions 41 may have rounded corners or may be any suitable shape.

Referring to FIGS. 3, 5-8, and 11, the adhesive portion or adhesive may comprise hydrogel. Hydrogels include a class of materials, generally comprising cross-linked polymeric chains dispersed in a continuous water phase, that are characterized by their ability to absorb a high mass fraction of water while remaining in a solid or gel state. The polymers used may be synthetic compounds such as polyvinyl alcohol, and naturally derived compounds such as agarose, collagen, or chitosan may be used. Their mechanical properties, for example elasticity and strength, can be controlled by varying the polymer content or degree of cross-linking. This can be of use for medical applications where it is important to match the strength and elasticity of underlying tissue. Hydrogels can be highly biocompatible and are and area of active research for drug delivery and surgical glues. In wound care, hydrogel or hydrocolloid materials may be used as a dressing material directly applied to a wound. The hydrogel may help absorb wound exudate, but also maintain some moisture at the wound to promote healing. Hydrogel adhesives are used for various medical applications such as defibrillation electrodes, transdermal drug delivery, and electrosurgical grounding pads. Hydrogel adhesives adhere well to skin even in the presence of water, hair or dirt, and can be removed without residue. Hydrogel adhesives are also used in military applications to temporarily secure explosives to targets, for example with breacher's tape. These hydrogel adhesives adhere well to many types of surfaces even when wet. The hydrogel adhesive 32 may provide strong adhesion to skin surrounding a wound 24 in spite of water or debris, and may be later removed without damaging the skin.

Referring to FIGS. 4-5 and 9-10, if a wound dressing system comprises a bandage, it may also comprise an anchoring feature for securing the free end (tail end 14B) of the bandage 14 once the bandage 14 has been wrapped around a patient's body or limb. The anchoring feature may, for example, be an adhesive patch, a clip that permits the free end of the bandage to be clipped to a wrapped bight of the bandage, or a patch of hook-and-loop material. If no anchoring feature is provided or one is provided and it fails, a medic may use a knot to secure the free end of the bandage.

Referring to FIGS. 4-5 and 9-10, the bandage 14 may comprise an adhesive tail portion 41. The portion 41 may comprise a suitable adhesive such as a hydrogel. The tail portion 41 may have a suitable structure, such as if the portion 41 has a multilayer form. The portion 41 may have a backing layer 94, such as for mounting hydrogel. The layer 94 may form the base of the portion 41. The portion 41 may have an adhesive layer 92, such as comprising hydrogel or another suitable solid state adhesive, for example seated upon layer 94. A sacrificial liner layer 90 may be located on a nose 92D of layer 92, leaving the base 92E of the layer 92 exposed to higher layers. The layer 90 may be oriented release side down. The layer 90 may form an example of a tab for a release liner or removable outer tail portion cover 88. The tail portion cover 88 may be seated upon the layers 90 and 92. The cover 88 may be made of a suitable material, such as polyethylene or another reduced adherence material to permit convenient and effective removal of the cover 88 from the adhesive layer 92. The layers may have a suitable shape, for example a rounded edge square shape for bases 88E, 92E, and 94E, with rounded edge triangular noses 84D, 90D, 92D, and 94D.

The tail end 14B may be secured using other methods, for example using a hook-and-loop connector, a C-clip 48 as shown in FIG. 19, a dowel, or others. Relative to the aforementioned, an adhesive tail portion 41 may be lighter and more easily packaged, less susceptible to breaking, applicable without fine motor skills, and resistant to water, temperature, and debris, especially it comprises a retention hydrogel 46.

Referring to FIGS. 5, 9-10, and 13, the bandage 14 may be connected to the dressing 12 via a detachable connector 50, which may allow the bandage 14 to be quickly separated from the dressing 12. The detachable connector 50 may, for example, be a frangible portion of the bandage 14. The connector 50 may be a hook and loop connector (not shown). Referring to FIG. 13, in some cases connector 50 may comprise a portion of the bandage 14 that is structured to be severed, for example using sufficient opposed elongate force or using a tool such as scissors 51. The connector 50 may comprise other ways of severing, such as score or line of perforations to predispose material for tearing, or an adhesive. Once detached, the bandage 14 may be discarded or used for a different application, for example to address a strain or to compress packing or another dressing against a different wound. Thus, a wound dressing system 10 may comprise a bandage 14 and a dressing 12 that are connected by a joint which is detachable, such that the bandage 14 and dressing 12 can be used independently. Such may be of advantage in combat scenarios where it is important to use versatile equipment and supplies may be limited.

Referring to FIGS. 5-10, 12-13, and 14A-14C, the bandage 14 may have a length sufficient to wrap at least once around a torso of a patient 22, for example if the bandage 14 has a length of at least forty eight inches. The bandage 14 may be more than sixty inches long. A relatively long length may allow the bandage 14 to, for example, encircle a patient's torso, such as a patient's chest 22A or waist 22E, or it may allow the bandage 14 to be used for applications other than securing a dressing 12, for example immobilizing a fractured bone. In the example of FIG. 14A, the bandage 14″ is sized to pass around the waist 22E and leg 22C of the patient 22, to support a relatively higher junctional injury or wound 24″.

Referring to FIG. 9, in some cases the bandage 14 comprises a dressing pad 86. The dressing pad 86 may be disposed on the bandage 14 somewhere between the dressing 12 and the ends 14A and 14B of the bandage 14. A bandage mounted dressing pad 86 and/or dressing may be useful to use with bandage 14 to secure a second wound, if the dressing 12 is in use on a separate wound 24 that does not require the bandage 14 to be used. In some cases a dressing pad 86 example may be advantageous if the bandage 14 is provided with a detachable connector 50. If the bandage 14 is detached from a dressing 12, for example when the dressing 12 comprises a peripheral adhesive portion 20 and thus can be secured to a wound without a bandage 14, then the bandage 14 may be left unused. If a dressing pad 86 is provided, then bandage 14 and dressing pad 86 may be used to treat another wound once separated from dressing 12.

Referring to FIGS. 6-11, the bandage 14 may be secured in an at least partially rolled configuration via a roll retention mechanism. The roll retention mechanism may comprise a plurality of stays, such as hook and loop connectors (not shown) spaced longitudinally along the bandage 14. The bandage 14 and system 10 may be provided in a rolled state, and it may be advantageous to control the unravelling of the bandage 14 to permit use of only the amount of length of bandage 14 that is desired to be used, keeping the remaining roll in a ravelled or rolled state. For example, when a dressing system includes an elastic bandage, it may be spirally rolled, and the roll may comprise retention features that prevent accidental unspooling if the roll is dropped. For example, hook-and-loop patches longitudinally positioned along the length of the elastic bandage may act as stays and prevent unravelling of successive layers of the spool.

Referring to FIGS. 6-11, the roll retention mechanism may comprise a thread 58 or other elongate member. The thread 58 may extend perpendicular relative to a roll axis 60 of the bandage 14 in the at least partially rolled configuration. The thread 58 may comprise adhesive to secure the thread 58 to plural layers of rolled bandage 14. A thread or yarn may thus be used as a retention feature by passing such through successive layers within the rolled bandage, and anchored in the first and last layers of bandage by a knot or adhesive. Such provides some resistance to unravelling that can be overcome by a person but not by the weight of the roll alone. The thread 58 may be a waxed cotton thread, that may be passed perpendicularly relative to a central axis 60 of the bandage 14 in the at least partially rolled configuration. The thread 58 may thus secure successively wrapped layers of the bandage 14 together, but in such a way that the layers can be easily separated purposefully by a user. The thread 58 may comprise a knot 98 or other to secure a thread end of the thread to the bandage 14.

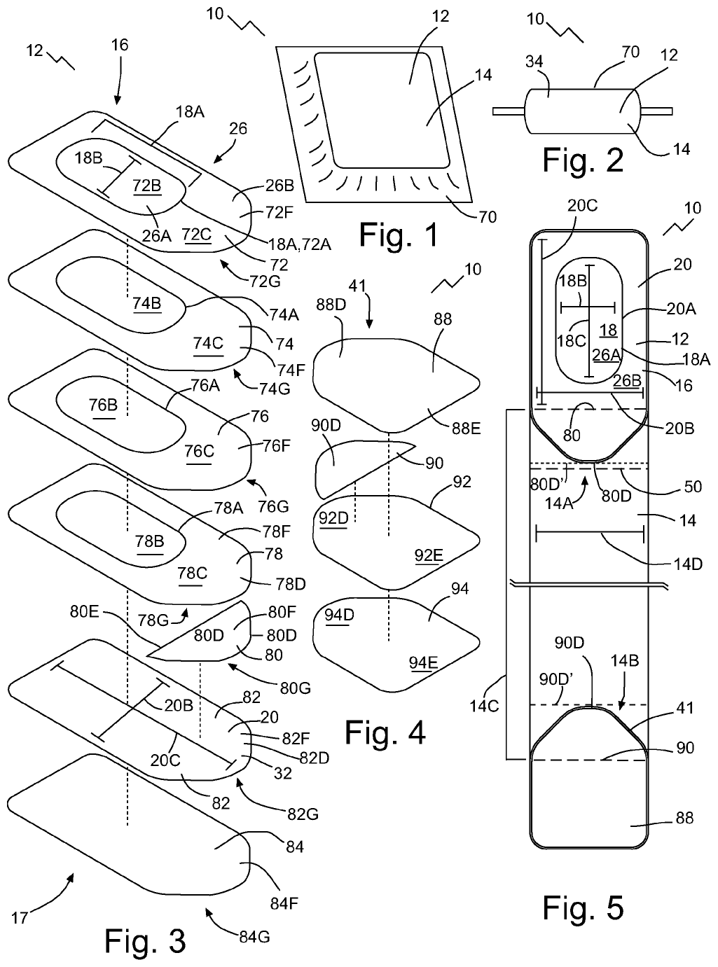

Referring to FIGS. 1 and 2, the wound dressing system 10 may be stored and transported in a suitable configuration, for example the system 10 may comprise an outer packaging 70 enclosing the dressing 12 and if present the bandage 14. The outer packaging may comprise vacuum packaging, for example to provide a compact, easy to store, light packaging. A wound dressing system 10 may be packaged in plastic vacuum-sealed wrappers so that such is compact and lightweight, and to prolong the shelf life of for example, plastic and adhesive materials. The outer packaging 70 may serve to protect the wound dressing system 10 from oxygen, ultraviolet light, moisture, and debris, and to maintain sterility. The outer packaging 70 may also keep the wound dressing system 10 small and compact for easy carriage in combat or by an emergency responder.

Referring to FIGS. 6-10 and 12-13, and 14A-C, several example methods of use are illustrated. FIGS. 6-7 illustrate a method of removing outer cover 28, unrolling the roll 34 of bandage 14, and removing the cover liner layer 90 of the tail portion 41. Referring to FIGS. 12-13 and 14A-C, once the cover 28 is removed, the interior portion 18 is ready to be secured against the wound 24. The bandage 14 may then be wrapped one or two times around the patient 22. in the example shown in FIGS. 12-13 and with dressing systems 10″ and 10′″ in FIG. 14A, the method of FIGS. 6-10 is carried out, and the tail portion 41 secured after wrapping the bandage 14 around the patient's upper leg 22C and waist 22E (system 10″), or wrapping the bandage 14 around the patient's leg 22C (system 10′″). Such could also be carried out to address wounds on the patient's arms 22B, neck 22F, and groin 22D. Referring to FIGS. 12-13 and 14A, in some cases, such as for relatively large wounds 24′ (system 10′), the bandage 14 may be removed (or may never have been present), either by using scissors 51 or by applying force to sever the bandage 14 at the connector 50. For a groin 22D wound 24, it may be advantageous to provide a relatively long bandage 14, to permit the system 10 to be mounted on the pelvis without sliding. Referring to FIG. 14B, an embodiment is shown similar to wound 24′ in FIG. 14A, except where the bandage 14 is not removed from the dressing 12. In such an embodiment, the dressing 12 is adhered over the wound 24, the bandage 14 extended several times around the chest 22A of the patient (below the arms 22B), and then crossed around the back of the patient 22 over the shoulder of the patient. The adhesive tail portion 41 may secure the end 14B of the bandage 14 to the patient 22 or to the system 10. Referring to FIG. 14C, an embodiment is shown where the dressing 12 is applied to a neck wound 24. In such a case the bandage 14 may be removed or used. In the example shown the bandage 14 is wrapped with a first section 14E′ around the neck 22F of the patient 22. Next, the bandage 14 is wrapped across the patient's back to a second section 14E″ that starts under the left armpit and wraps several times around the patient's upper chest 22A. Finally, the bandage 14 is wrapped across the back of the patient 22 to a third section 14E′″ that crosses over the left shoulder of the patient 22 and down across the chest 22A. The tail portion 41 may secure the end 14B of the bandage to the patient 22 or system 10. The examples in FIGS. 14A-C are intended to be exemplary to show various ways of applying the system 10 to a patient, and are not intended to be limiting.

Referring to FIGS. 15, a vent 36 may be defined in the dressing 12. A vent 36 may be used to address tension or traumatic pneumothorax. A tension pneumothorax is a life-threatening condition that develops when air is trapped in the pleural cavity under positive pressure, displacing mediastinal structures and compromising cardiopulmonary function. Because tension pneumothorax occurs infrequently and has a potentially devastating outcome, a high index of suspicion and knowledge of basic emergency thoracic decompression procedures are important for all healthcare personnel. Immediate decompression of the thorax is mandatory when tension pneumothorax is suspected. This should not be delayed for radiographic confirmation. Traumatic open pneumothorax can be treated by applying an occlusive wound dressing, a wound dressing that creates a complete fluid seal around the perimeter of the wound and permits air ingress. The occlusive wound dressing may be equipped with a one-way valve that permits air egress. This can reduce the risk of tension pneumothorax and secondary tissue damage. An occlusive bandage is usually attached to the skin with an adhesive layer or by wrapping with an elastic bandage. Occlusive bandages in combination with absorbent pads may also serve to maintain moisture level within a wound site by absorbing excess moisture or by preventing evaporative moisture loss. Conserving moisture at the wound site may prevent the formation of a scab and consequent pain or slow healing.

Some chest wounds can cause traumatic pneumothorax, a condition where air is allowed to abnormally accumulate in the pleura, the lining of the lungs. In the case of open pneumothorax, a chest wound such as a bullet wound may have opened a passage from the lungs to the environment. A complication called a tension pneumothorax can further develop when damaged tissue acts as a one-way valve from the lungs to the chest cavity, which can lead to a shortage of oxygen in the blood. The use of a vent 36, for example an adjustable vent that can be opened or closed by the user, may be advantageous to prevent the dressing 12 from functioning as a full occlusive dressing 12.

Referring to FIGS. 15-19, various embodiments of dressings 12 are illustrated. Referring to FIG. 14A, a rounded edge rectangular shaped dressing 12 is illustrated, with opposed tabs 30, a vent 36, an adhesive portion 20, and an extra dressing pad 86 on the bandage 14. Referring to FIG. 16, a square contacting portion 18 is surrounded by a circular adhesive portion 20, including a tab 30 off the lateral edge of the cover 28. Referring to FIG. 17, a square interior portion 18 is surrounded by an oblong, oval portion 20, with opposed tabs 30 and an extra dressing pad 86 on the bandage 14. Referring to FIG. 18, square portions 18 and 20 are shown, with stacked tabs 30. Referring to FIG. 19, an embodiment is illustrated where the bandage 14 has an anti-roll C-clip 48.

Immaterial modifications may be made to the embodiments described here without departing from what is covered by the claims.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

1. A wound dressing system comprising:
a bandage; and
a dressing forming a patient-contacting face that defines an interior wound-contacting portion and a peripheral adhesive portion, the peripheral adhesive portion at least partially surrounding the interior wound-contacting portion, with a removable outer cover overlying the peripheral adhesive portion comprising a dressing pad.

2. (canceled)

3. The wound dressing system of claim 1 in which the interior wound-contacting portion comprises a dressing pad.

4. (canceled)

5. The wound dressing system of claim 1 in which the dressing is formed by:
an adhesive layer defining the peripheral adhesive portion;
a removable outer cover layer defining the removable outer cover; and
a dressing pad layer overlying the removable outer cover layer;
in which the removable outer cover layer and the dressing pad layer are cut along aligned interior paths to separate the removable outer cover layer and the dressing pad layer into interior and peripheral portions, with the interior portion of the dressing pad layer defining the interior wound-contacting portion, and the peripheral portion defining a peripheral wound-contacting portion.

6. The wound dressing system of claim 1 in which the peripheral adhesive portion comprises hydrogel.

7-8. (canceled)

9. The wound dressing system of claim 1 in which the removable outer cover comprises a tab.

10. The wound dressing system of claim 1 in which the interior wound-contacting portion has a stadium shape.

11. (canceled)

12. The wound dressing system of claim 1 in which the bandage is sized to extend at least one revolution around a patient's torso.

13. The wound dressing system of claim 12 in which the bandage has a length of at least forty eight inches to sixty inches.

14. (canceled)

15. The wound dressing system of claim 1 in which the bandage has a detachable connector that secures the bandage to the dressing in which the detachable connector comprises a frangible portion of the bandage, such as a hook and loop connector.

16-17. (canceled)

18. The wound dressing system of claim 1 in which the bandage comprises a dressing pad.

19. The wound dressing system of claim 1 in which the bandage is secured in an at least partially rolled configuration via a roll retention mechanism.

20. The wound dressing system of claim 19 in which the roll retention mechanism comprises a plurality of stays spaced longitudinally along the bandage, wherein the plurality of stays are preferably hook and loop connectors.

21. (canceled)

22. The wound dressing system of claim 19 in which the roll retention mechanism comprises a thread, wherein the thread preferably extends perpendicular relative to a roll axis of the bandage in the at least partially rolled configuration.

23. (canceled)

24. The wound dressing system of claim 22 in which the thread comprises adhesive.

25-26. (canceled)

27. The wound dressing system of claim 1 in which the adhesive tail portion has a removable outer tail portion cover, wherein the removable outer tail portion cover preferably comprises a tab.

28. (canceled)

29. The wound dressing system of claim 1 further comprising an outer packaging enclosing the bandage and dressing portion, preferably vacuum packaging.

30. (canceled)

31. The wound dressing system of claim 1 in which the interior wound-contacting portion is sized to cover a bullet wound having a minimum lateral dimension of two inches or more.

32-35. (canceled)

36. A wound dressing system, comprising:
a dressing forming a patient-contacting face that defines an interior wound-contacting portion and a peripheral adhesive portion, the peripheral adhesive portion at least partially surrounding the interior wound-contacting portion, with a removable outer cover overlying the peripheral adhesive portion, the removable outer cover having a dressing pad.

37-39. (canceled)

40. The wound dressing system of claim 36 wherein
the peripheral adhesive portion comprising hydrogel and the interior wound-contacting portion comprises a non-adherent gauze dressing pad.

41. A wound dressing system comprising:
a bandage; and
a dressing having:
an adhesive layer;
a removable outer cover layer overlying the adhesive layer; and
a dressing pad layer overlying the removable outer cover layer;
in which the removable outer cover layer and the dressing pad layer are cut along aligned interior paths to separate the removable outer cover layer and the dressing pad layer into interior and peripheral portions.
